(19) United States
(12) Patent Application Publication
(10) Pub. No.: US 2010/0152551 A1
(43) Pub. Date: Jun. 17, 2010

(54) INTEGRATED MEDICAL MEASUREMENT SYSTEM UTILIZING INFRARED TRANSMISSION

(76) Inventors: Tien-Tsai HSU, Hsinchu (TW); Chi-Chang Nien, Taipei (TW)

Correspondence Address: Muncy, Geissler, Olds & Lowe, PLLC, 4000 Legato Road, Suite 310, FAIRFAX, VA, 22033, US

(21) Appl. No.: 12/635,185

(22) Filed: Dec. 10, 2009

(30) Foreign Application Priority Data
Dec. 12, 2008 (TW) 97148553

(51) Int. Cl. A61B 5/00

(52) U.S. Cl. 600/301

(57) ABSTRACT

An integrated medical measurement system utilizing infrared transmission comprises a medical record device and a plurality of barcodes. The medical record device has a microprocessor connected with a barcode scanner. The barcode scanner is used to scan the barcodes worn on patients. A first measurement device connected with the microprocessor is used to detect the patients' physiological data values. A first infrared transmission device is connected with the microprocessor and it is also connected with a second infrared transmission device of a second measurement device wirelessly, such that the medical record device can receive the data from the second measurement device.

### BACKGROUND OF THE INVENTION

1. Field of the Invention

The present invention relates to a medical measurement system, and in particular to an integrated medical measurement system having a barcode scanner utilizing infrared transmission.

2. Description of the Related Art

The prior art of patent number M242181 of Taiwan patent discloses a data storage device integrated with various medical measurement devices, which includes a housing and a controller unit. The controller unit installed in the housing is used to control the operations of the various units. A transmission unit connected to the controller unit via an electrical wire is used to receive data from an external measurement device and send the data to the controller unit. A data storage unit, connected to the controller unit electrically, is used to store the data received from the transmission unit. A display unit is connected to a controller unit electrically, and is used to display data. A calculation unit is used to process and compare data with the expected value advised by a doctor. A warning unit connected to the controller unit electrically is used to send out an alarm signal.

In the prior art, the data storage device integrated with the various medical measurement devices can not identify data from the different patients efficiently. When a medical attendant takes a plurality of patients' physiological data values, he or she must distinguish the patients by manually controlling the medical measurement device. Alternatively, the medical attendant can take note and record every patient's measurement results on paper, and then input the results into a computer of the nursing station. Both ways mentioned above are very time-consuming and error prone.

Therefore, the present invention provides an integrated medical measurement system utilizing infrared transmission to overcome the shortcoming of the prior art.

### SUMMARY OF THE INVENTION

A primary objective of the present invention is to disclose an integrated medical measurement system utilizing infrared transmission. The restriction of distance and direction of infrared transmission allows the signal not to be interfered. The infrared transmission has the advantage of convenience of wireless transmission but doesn't exist the shortcoming of mutual interference of the wireless transmission.

Further, a barcode scanner is utilized to verify patients' and operators' identifications, and a first infrared transmission device is utilized to receive the data and the patients' physiological data values from a second measurement device. Both kinds of the above mentioned data are stored in a medical record device. A USB slot is utilized to connect the medical record device with a computer in a nursing station.

Also, the computer in the nursing station is connected with a Hospital Information System (HIS) or a Laboratory Information System (LIS) through TCP/IP, such that, the data related to the patients can be fully utilized.

To achieve the above mentioned objective, the present invention provides an integrated medical measurement system utilizing infrared transmission, which comprises a medical record device and a plurality of barcodes. The medical record device comprises a microprocessor connected to a barcode scanner. The barcode scanner is used to scan the identification barcodes worn on the patients and the operators. A first measurement device connected to the microprocessor is utilized to detect every patient's physiological data values. A first infrared transmission device is connected to the microprocessor for receiving data from the second measurement device. In the present invention, barcode scanner is used to verify the identity of a patient, and the first infrared transmission device can receive the data and the patients' physiological data values transmitted from the second measurement device. The above mentioned data is stored in the medical record device, which is connected with a computer in a nursing station through a USB slot. The characteristic of the infrared transmission is that its distance and direction can be restricted, thus avoiding the signal interference or the transmission error. Also, the computer in the nursing station is connected with a Hospital Information system or a Laboratory Information System through TCP/IP, such that data related to the patients can be fully utilized.

It is to be understood that both the foregoing general description and the following detailed description are exemplary, and are intended to provide further explanation of the invention as claimed.

### BRIEF DESCRIPTION OF THE DRAWINGS

FIG. 1 is a block diagram of an integrated medical measurement system utilizing infrared transmission according to an embodiment of the present invention; and

FIG. 2 is a block diagram of a medical record device according to an embodiment of the present invention.

### DETAILED DESCRIPTION OF THE INVENTION

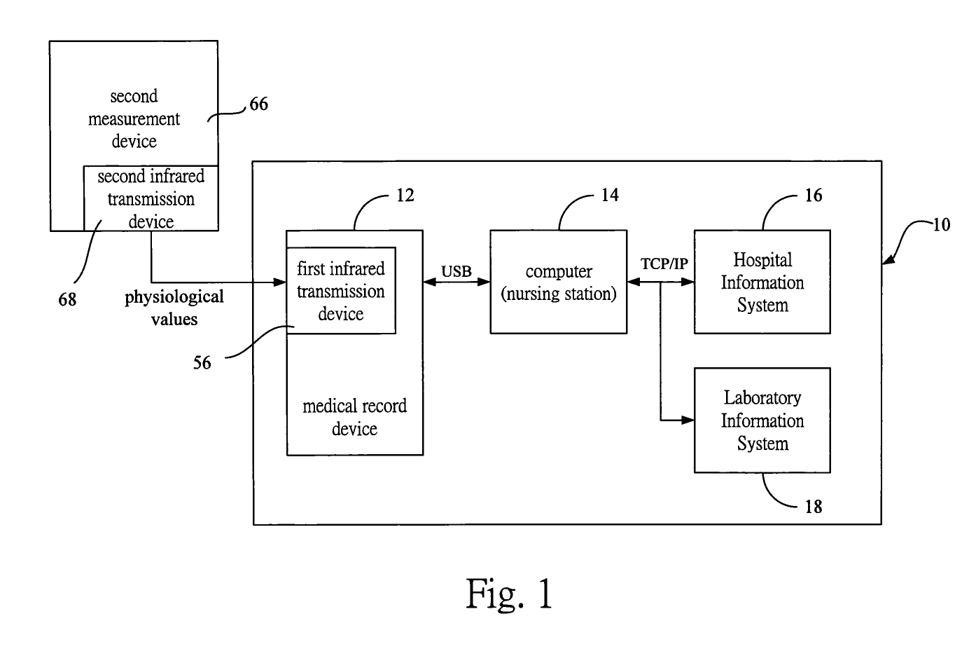

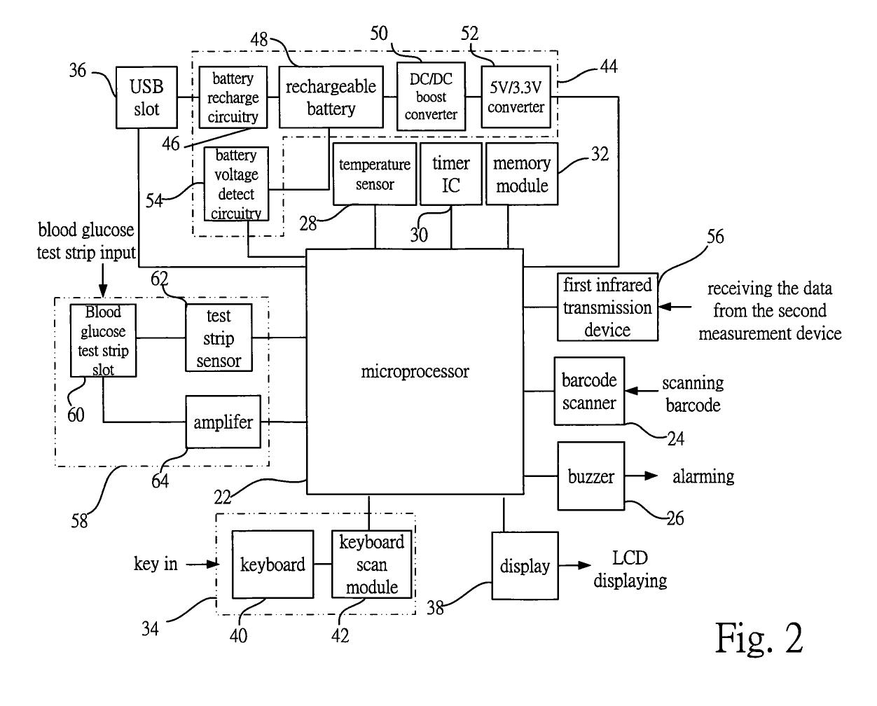

Refer to FIG. 1 and FIG. 2 in order to explain the present invention in detail. In a hospital, patients are provided with barcodes representing their identification data. An integrated medical measurement system utilizing infrared transmission 10 according to the present invention comprises a medical record device 12 and barcodes. The number of the barcodes on each of the patients is not limited. The medical record device 12 comprises a microprocessor 22 connected with a barcode scanner 24, a buzzer 26, a temperature sensor 28, a timer IC 30, a memory module 32, a keyboard input module 34, a USB slot 36 and a display 38 respectively. The barcode scanner 24 is utilized to scan the barcodes worn on the patients. As a result, when a medical attendant uses the barcode scanner 24 to scan the barcode, he or she can identify a patient A (a specific patient) through the medical record device 12. Moreover, the keyboard input module 34 comprises a keyboard 40 and a keyboard scan module 42. In case that there is no barcode for the patients or the barcode scanner is not working, the medical attendant can input the identifications of the patients and the medical attendant and the data related to the patients into the medical record device 12 through a keyboard 40. The data related to patient A is shown on a display 38.

A power system 44 located between the USB slot 36 and the microprocessor 22 comprises a battery recharge circuitry 46, a rechargeable battery 48, a DC/DC boost converter 50, a 5V/3.3V converter 52 and a battery voltage detect circuitry 54. The two ends of the battery recharge circuitry 46 are connected respectively with the USB slot 36 and the rechargeable battery 48; the two ends of the DC/DC boost converter 50 are connected respectively with the rechargeable battery 48 and the 5V/3.3V converter 52; the other end of the 5V/3.3V converter 52 is connected to the microprocessor 22; the battery voltage detect circuitry 54 is located between the rechargeable battery 48 and the microprocessor 22. The power system 44 recharged through the USB slot 36 supplies the medical record device 12 with the electrical power. The rechargeable battery 48 can be a lithium battery.

A first infrared transmission device 56 is connected with a microprocessor 22. A first measurement device 58 connected with a microprocessor 22 is used to detect the physiological data values of the patients. In one embodiment of the present invention, the first measurement device 58 in FIG. 2 is a blood glucose meter. It could be also a thermometer, a blood pressure meter, an oximeter, a pulsimeter, a weight scale, a body fat meter, or other devices which is used to detect the other physiological data values.

At least one second measurement device 66 located outside the medical record device 12 comprises a second infrared transmission device 68. By connecting the medical record device 12 with the second infrared transmission device 68 electrically through a first infrared transmission device 56, the medical record device 12 receives the data from the second measurement device 66. The above mentioned data is stored temporarily in the medical record device 12, and then the data is sent to a computer 14 in the nursing station through the USB slot 36. The second measurement device 66 can also be a kind of device capable of detecting the physiological data values, such as a thermometer, a blood pressure meter, a blood glucose meter, an oximeter, a pulsimeter, a weight scale, or a body fat meter.

According to the differences of the above mentioned measurement devices, the physiological data value could be a blood pressure value, a body temperature value, a blood glucose value, a blood oxygen value, a pulse value, a weight value or a body fat value.

In the embodiment of the present invention, the first measurement device 58 is the blood glucose meter including a blood glucose test strip slot 60, a test strip sensor 62, and an amplifier 64. The blood glucose test strip is detected by the test strip sensor 62 through the blood glucose test strip slot 60. The signal of the blood glucose is transmitted to the microprocessor 22 after the signal is amplified by the amplifier 64. The blood glucose value of patient A obtained through calculation is stored into the memory module 32.

Besides, when the first infrared transmission device 56 in the medical record device 12 or the second infrared transmission device 68 in the second measurement device 66 breaks down or malfunctions, the medical attendant still can input the data into the medical record device 12 through the keyboard input module 34, and the data such as a blood pressure value, a body temperature value, a blood glucose value, a blood oxygen value, a pulse value, a weight value or a body fat value, is measured by the second measurement device 66.

Because the barcode scanner 24 has scanned/inputted the data of the patient while, and the medical record device 12 has recorded the patient's data and time of recording the data, the medical attendant only needs to input another physiological data value acquired from the second measurement device 66. Compared with the prior art, the different patients are distinguished by controlling the medical measurement device manually or taking a note with a paper, the embodiment of the present invention is more convenient and has lower error possibilities.

The medical record device 12 is able to record the data of a plurality of the patients. The medical attendant can use the USB slot 36 to output the data into the computer 14 in the nursing station. The computer 14 is connected to a Hospital Information System (HIS) 16 or a Laboratory Information System (LIS) 18 through TCP/IP.

The barcode scanner 24 is utilized to identify the identifications of the patients according to one embodiment of the present invention. The first measurement device 58 (the blood glucose meter) is utilized to detect the blood glucose value of the patient. The first infrared transmission device 56 is connected wirelessly with the second infrared transmission device 68 of the second measurement device, such that the medical record device 12 can receive the data from the second measurement device 66, and the data can be stored in the medical record device 12, and that is connected to a computer 14 in a nursing station through a USB slot 36. Due to the characteristic of the infrared transmission, the restrictions of its distance and direction ensure that signal will not be interfered. Also, TCP/IP is utilized to transmit data from the computer 14 in the nursing station to the Hospital Information System 16 or the Laboratory Information System 18, such that the data related to the patients can be fully utilized. As a result, the medical record device 12 is able to achieve the functions of scanning barcode, recording and transmitting the data of the patients. All the related data (body temperature, blood pressure, blood glucose, blood oxygen, pulse and so on) of the patients are transmitted to the nursing station and even to the Hospital Information System 16 or to the Laboratory Information System 18 located in the back end of the nursing station, such that the present invention could achieve the objective of the comprehensive medical record digitalization.

Those described above are only the preferred embodiments to clarify the technical contents and characteristics of the present invention in enabling the persons skilled in the art to understand, make and use the present invention. However, they are not intended to limit the scope of the present invention. Any modification and variation according to the spirit of the present invention can also be included within the scope of the claims of the present invention.

1. An integrated medical measurement system utilizing infrared transmission, comprising:
at least one barcode and a medical record device, said barcode is worn on at least one patient and said medical record device comprises:
a microprocessor;
a barcode scanner, connected with said microprocessor, and is used to scan said barcode worn on said patient;
a keyboard input module and a display, connected respectively with said microprocessor;
a first infrared transmission device, connected with said microprocessor; and
a first measurement device, connected with said microprocessor, is used to detect a physiological data value of said patient, and store data related to said physiological data value in said medical record device according to said barcode worn on said patient; and
at least one second measurement device, is used to detect a physiological data value of said patient, comprises a second infrared transmission device, and said second infrared transmission device is wirelessly connected with said first infrared transmission device.

2. The integrated medical measurement system utilizing infrared transmission according to claim 1, wherein said first measurement device is selected from a group consisting of: a blood pressure meter, a thermometer, a blood glucose meter, an oximeter, a pulsimeter, a weight scale, or a body fat meter.

3. The integrated medical measurement system utilizing infrared transmission according to claim 1, wherein said second measurement device is selected from a group consisting of: a blood pressure meter, a thermometer, a blood glucose meter, an oximeter, a pulsimeter, a weight scale, or a body fat meter.

4. The integrated medical measurement system utilizing infrared transmission according to claim 1, wherein said medical record device receives said physiological data value, such as a body temperature value, a blood glucose value, a blood oxygen value, a pulse value, a weight value or a body fat value, from said second measurement device by wirelessly connecting said second infrared transmission device with said first infrared transmission device.

5. The integrated medical measurement system utilizing infrared transmission according to claim 1, wherein said physiological data value, such as a blood pressure value, a body temperature value, a blood glucose value, a blood oxygen value, a pulse value, a weight value or a body fat value, detected by said second measurement device is inputted into said medical record device through said keyboard input module.

6. The integrated medical measurement system utilizing infrared transmission according to claim 1, wherein said keyboard input module comprises a keyboard scan module and a keyboard.

7. The integrated medical measurement system utilizing infrared transmission according to claim 1, further comprising: a buzzer, a temperature sensor, a timer IC and a memory module that are respectively connected with said microprocessor.

8. The integrated medical measurement system utilizing infrared transmission according to claim 1, further comprising: a USB slot connected with said microprocessor for providing updated data.

9. The integrated medical measurement system utilizing infrared transmission according to claim 8, wherein a power system is located between said USB slot and said microprocessor, and said power system is recharged through said USB slot.

10. The integrated medical measurement system utilizing infrared transmission according to claim 9, wherein said power system comprises:
a battery recharge circuitry, one end of said battery recharge circuitry is connected to said USB slot;
a rechargeable battery, one end of said rechargeable battery is connected to an other end of said battery recharge circuitry;
a battery voltage detect circuitry, is located between said rechargeable battery and said microprocessor;
a DC/DC boost converter, an end of said DC/DC boost converter is connected to an other end of said rechargeable battery; and
a 5V/3.3V converter, one end of said 5V/3.3V converter is connected to an other end of said DC/DC boost converter, and an other end of said 5V/3.3V converter is connected to said microprocessor.

11. The integrated medical measurement system utilizing infrared transmission according to claim 10, wherein said rechargeable battery is a lithium battery.

12. The integrated medical measurement system utilizing infrared transmission according to claim 8, wherein said medical record device is connected with a computer through said USB slot, and said computer is connected with a Hospital Information System (HIS) or a Laboratory Information System (LIS) through TCP/IP.

13. The integrated medical measurement system utilizing infrared transmission according to claim 2, wherein said blood glucose meter comprises a blood glucose test strip slot, a test strip sensor, and an amplifier.
